(19) United States
(12) Patent Application Publication
(10) Pub. No.: US 2025/0032943 A1
(43) Pub. Date: Jan. 30, 2025

(54) SOFT TACTILE ARTICLE FOR THERAPEUTIC USE

(71) Applicant: Turbo Relief LLC, Greenwich, CT (US)

(72) Inventors: Ryan Miele, Greenwich, CT (US); Mihir Keskar, Beaverton, OR (US)

(21) Appl. No.: 18/785,684

(22) Filed: Jul. 26, 2024

Related U.S. Application Data
(60) Provisional application No. 63/529,222, filed on Jul. 27, 2023.

(51) Int. Cl. A63H 3/00; A61M 21/00; A61M 21/02; A63H 3/02

(52) U.S. Cl. CPC A63H 3/003; A61M 21/02; A63H 3/02; A61M 2021/0016; A61M 2021/0022; A61M 2021/0066

(57) ABSTRACT

A therapeutic device including a main body, one or more components extending from the main body, and one or more sub-components extending from the one or more components. At least one of the main body, the one or more components and the one or more sub-components are stuffed with a filling. A cooling feature is disposed at or within the one or more components or the one or more sub-components. A tactile feature is disposed at or within the one or more components or the one or more sub-components. An aromatherapy feature is disposed at or within one or more of the components or the one or more sub-components. The main body is configured to provide weighted pressure to a user of the device.

### RELATED APPLICATION

This application is a nonprovisional based on and claiming priority to U.S. Provisional Application No. 63/529,222, filed Jul. 27, 2023 and entitled SOFT TACTILE ARTICLE FOR THERAPEUTIC USE, the contents of which are incorporated herein by reference in their entirety.

### FIELD

The present invention generally relates to soft tactile articles, such as, for example, plush toys, for therapeutic use.

### BACKGROUND

There is a large, underserved need for an improved focus on mental health for the youth, including young adolescent children both without and with intellectual disabilities. In addition to the mental health needs of the youth community largely being unmet, very few seek help because of the ever-persistent stigma around receiving help for a mental issue. Mental health issues, such as, for example, stress, anxiety, and depression can be addressed with therapy and/or drug intervention. However, children and young teens often do not have access to mental health counselors and/or are hesitant to seek help due to stigmatization of such services, and drugs often have undesirable side effects and/or are cost prohibitive. Accordingly, there is a need for cost-effective physical device which provides drug-free mental relief and works to destigmatize the use of mental health products.

### SUMMARY

An object of the present invention is to provide a therapeutic device that reduces anxiety and improves mental health in children, young teenagers and elderly adults.

Another object of the present invention is to provide a therapeutic device for everyone, including young children, pre-adolescents, adolescents and elderly adults suffering from intellectual disabilities such as anxiety, dementia or Alzheimer's, that physically alleviates the negative mental effects brought about by daily interactions with the world around them.

A therapeutic device according to an exemplary embodiment of the present invention comprises: a main body configured to provide weighted pressure to a user of the device; one or more components extending from the main body; one or more sub-components extending from the one or more components, at least one of the main body, the one or more components and the one or more sub-components being stuffed with a filling; a cooling feature disposed at or within the one or more components or the one or more sub-components; a tactile feature disposed at or within the one or more components or the one or more sub-components; and an aromatherapy feature disposed at or within one or more of the components or the one or more sub-components.

In an exemplary embodiment, the cooling feature comprises a cool pack or an ice pack.

In an exemplary embodiment, the tactile feature comprises a patch of rough material.

In an exemplary embodiment, the tactile feature comprises beads.

In an exemplary embodiment, the aromatherapy feature comprises one or more essential oils.

In an exemplary embodiment, the therapeutic device is a stuffed toy.

In an exemplary embodiment, the stuffed toy resembles an animal comprising at least one of a body, head, cars, legs, paws and tail.

In an exemplary embodiment, the cooling feature is disposed within one of the ears.

In an exemplary embodiment, the tactile feature is disposed within the tail.

In an exemplary embodiment, the tactile feature is disposed at one of the ears.

### DESCRIPTION OF THE DRAWINGS

Exemplary embodiments of the present invention will be described with references to the accompanying figures, wherein:

FIGS. 1-4 show various perspective views of a therapeutic device according to an exemplary embodiment of the present invention.

### DETAILED DESCRIPTION

Stress and anxiety are effectively the emotions one feels when their body is presented with an issue and enters into their pre-historic “Fight or Flight” mentality. This mentality can result in an increased heart rate, blood pressure, tension in muscular extremities, and shortness of breath by activating one's sympathetic nervous system. Since one's body isn't actually in any physical danger, the brain doesn't know how to physically respond to the stressor in front of it at that moment. This is why if anyone has ever felt particularly anxious or stressed, they may feel almost frozen and physically tense or even begin to sweat. That's the body's natural physical response to the brain entering into that “Fight or Flight” mode. The key to reducing stress and anxiety is to find ways to alleviate one's physical symptoms thereby forcing your brain out of that “Fight or Flight” mentality allowing the individual to feel the sensation of safeness, calmness and collectivity.

A plush toy in accordance with exemplary embodiments of the present invention addresses symptoms associated with mental health issues, such as, for example, stress, anxiety and depression, by offering a combination of therapeutic components. In a sense, the inventive plush toy is the modern-day reinvention of the everyday stuffed animal that serves a therapeutic purpose. Every child embraces a stuffed animal in their youth for its cuddliness and instant companionship. In exemplary embodiments, the inventive plush toy takes advantage of this early access to those in their pre-adolescent years by immediately providing a mental health support system upon which its users can rely for the rest of their lives. The inventive plush toy provides an effective reactionary aid to those currently suffering from serious mental health stresses, and it also has value in its potential as a preventative device. It serves as an instant mental health support system introduced at a critical time in childhood development to help prevent mental health crises that are plaguing today's youth such as isolationism, self-harm, and suicide.

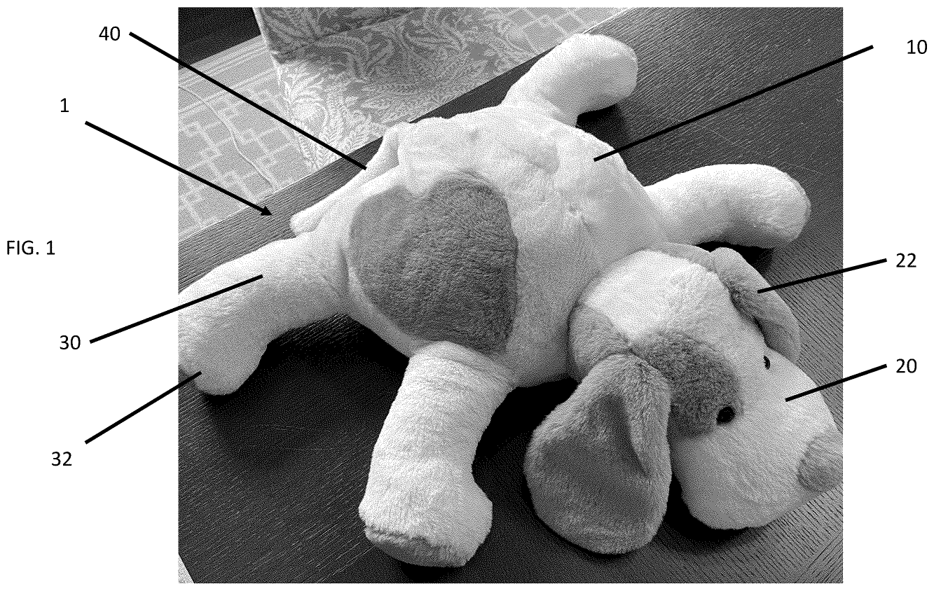

FIG. 1 is a perspective view of a therapeutic device, generally designated by reference number 1, according to an exemplary embodiment of the present invention. The therapeutic device 1 may be a plush toy, and in an exemplary embodiment the therapeutic device 1 is a plush toy in the form of a stuffed animal. The device 1 is shown in the figures as a stuffed toy dog, but it should be appreciated that the device 1 may be any other type of stuffed animal, such as, for example, a bear, a horse, a duck, a cat, etc. In exemplary embodiments, the device 1 may resemble a cartoon character, a legendary creature (e.g., unicorn) or an inanimate object (e.g., ball, block, etc.). In general, the device 1 is a soft and comfortable object that is intended to invoke feelings of calmness and safety. The device 1 may be constructed of outer fabrics such as plain cloth, or pile textiles like plush or terrycloth, and may be stuffed with, for example, synthetic fiber, batting, cotton, straw, wood wool, plastic pellets, and beans.

The therapeutic device 1 includes a main body 10 from which any number of components may extend. For example, as shown in the figures, the main body 10 corresponds to the main body of a dog, and the extending components include a head component 20, leg components 30 and a tail component 40. In exemplary embodiments, any number of sub-components may extend from each of the components. For example, ear sub-components 22 may extend from the head component 20, and a paw sub-component 32 may extend from each leg component 30.

In exemplary embodiments, the therapeutic device 1 includes a number of different features that offer clinically proven and psychiatric approved modes of anxiety reduction. In this regard, the device 1 may include a weighted body, and in particular the main body 10 may contain a filling that evenly distributes the weight and simulates the feeling of a warm blanket. When applied, weighted stuffed animals provide deep pressure touch that can reduce cortisol levels, place one's autonomic nervous system into “rest” mode making sensory processing easier and release feel-good brain chemicals like dopamine and serotonin. This weighted feature can help reduce anxiety, stress, impulsive behavior and increase one's ability to sit and concentrate on tasks. In exemplary embodiments, the device 1 may weigh 2 to 10 lbs or more, preferably 2 to 5 lbs, and in a preferred exemplary embodiments weights 5 lbs.

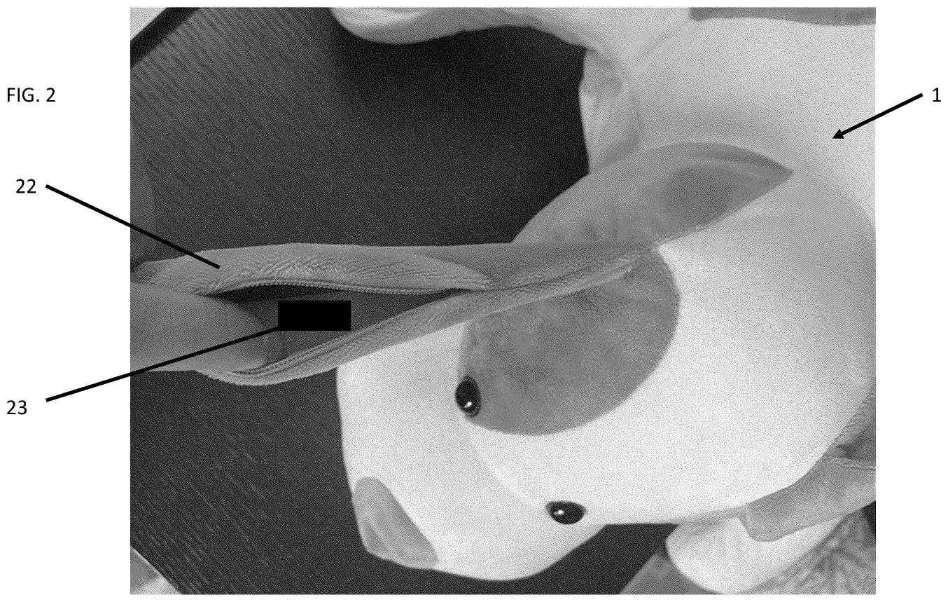

In an exemplary embodiment, one or more of the components or sub-components of the device 1 may include a cooling feature. For example, the cooling feature may be disposed in one or both ear sub-components 22 and/or one or more of the paw sub-components 32. In an exemplary embodiment, the cooling feature may be a cool pack or ice pack, filled with water, refrigerant gel, and/or liquid. In a specific exemplary embodiment shown in FIG. 2, the cooling feature is a non-toxic and biodegradable ice pack 23 disposed within a sleeve 24 formed in one of the ear sub-components 22 (for example, the right ear sub-component 22). The ice pack 23 may be circular in shape with a diameter in the range of, for example, 3 cm to 10 cm or more, and in a specific example, the diameter is 4 cm. The ice pack 23 may have any other shape besides circular, such as, for example, rectangular or triangular. The sleeve 24 may be opened and closed with a fastening element, such as, for example, a zipper or hook-and-loop fastener. In the embodiment shown in FIG. 2, the fastening element includes a zipper 25. When the ice pack 23 is not frozen, it may double as a squishable feature where the user can squeeze it providing another sensory relief aspect. When the user begins feeling especially anxious or stressed, including undergoing a panic attack, an ice pack can be removed from the freezer and placed inside the ear sub-component 22, providing a cold sensation. The science supporting this feature is as follows: placing the cold ear on the user's forehead activates the Vagus Nerve (the longest cranial nerve in the body connecting to multiple muscle groups throughout the body). In simple terms, this may sensation may be felt when diving into an ice bath or a cold swimming pool. Experts call this the “Dive Response.” This distracts one's mind from whatever they were thinking about earlier. This feeling of coldness causes blood to rush from non-essential to essential organs in addition to physically reducing one's heart rate. These sensations and physical effects combined give the physical feeling of calmness.

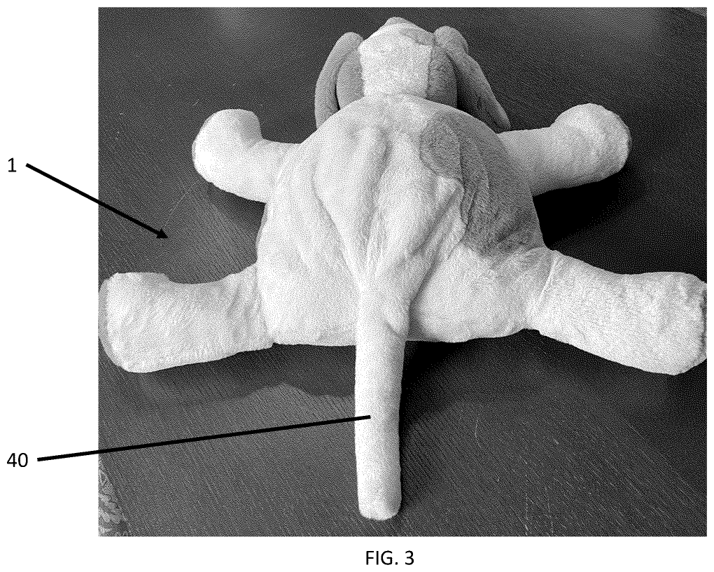

In an exemplary embodiment, one or more of the components or sub-components of the device 1 may include a tactile feature. For example, the tactile feature may be located at or in the head component 20, one or more of the paw-subcomponents 32 or the tail component 40. In an exemplary embodiment, the tactile feature may be beads disposed within one or more of the components or sub-components. In a specific exemplary embodiment shown in FIG. 3, the tactile component includes beads disposed within the tail component 40, the beads having sufficient porosity between them to allow a user to squeeze the tail component 40 and feel the beads move around within the tail component 40. The beads may be disposed within a packet within the tail component 40. Individuals may fidget or make small movements when they are feeling anxious, restless, impatient or nervous. The tactile feature including beads gives these movements an outlet, which can help calm a person's nerves, relieve stress and serve as a distraction in an overstimulating environment.

In exemplary embodiments, the cooling feature may also operate as a tactile feature, even if the cooling feature has not been activated to provide a cooling effect. For example, the cooling feature may be encased in an envelope or other type of enclosure so that it may function as a tactile massaging element when placed on the skin of a user.

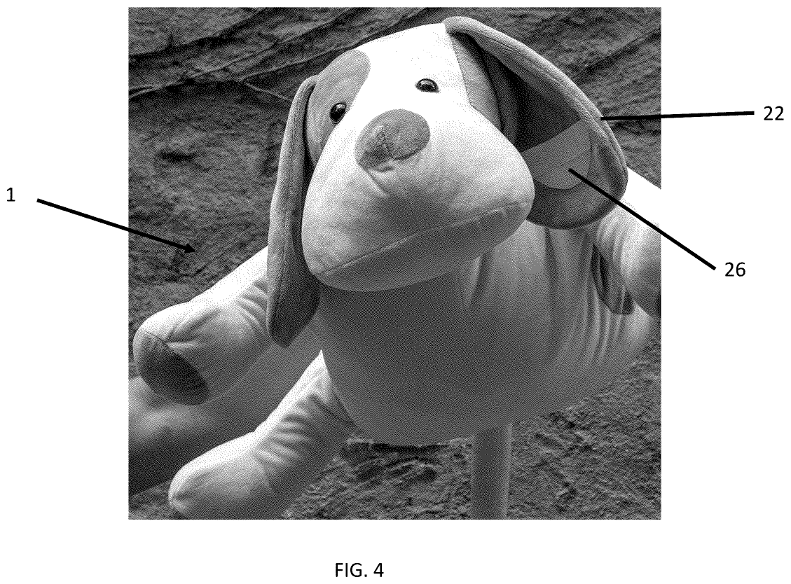

In another exemplary embodiment, the tactile feature may be a rough patch 26 disposed on one of the components or sub-components. In a specific exemplary embodiment shown in FIG. 4, the patch 26 includes a piece of hard rough Velcro® stitched or otherwise disposed on the underside of one of the ear sub-components 22. The patch 26 allows the user to grip and rub something hard and prickly providing a more intense sensory experience. Anxious fidgeting occurs because the body has elevated levels of stress hormones, which are prepping your muscles for sudden exertion. Fidgeting may be a self-regulation mechanism to help boost one's attention levels.

In an exemplary embodiment, one or more of the components or sub-components of the device 1 may include an aromatherapy feature. For example, any one or more of the components 10, 20, 30, 40 and/or sub-components 22, 32 may include a packet of a substance that emits a pleasant odor, such as, for example, lavender, rose, jasmine, rosemary, peppermint and lemon, to name a few. The substance may be, for example, one or more essential oils. In general, aromatherapy is the practice of using essential oils for therapeutic benefit and has been used for centuries as a means to reduce anxiety. Aroma immediately stimulates the central nervous system, triggering an emotional response and when inhaled, the scent molecules in essential oils travel from the olfactory nerves directly to the brain and especially impact the amygdala, the emotional center of the brain.

In exemplary embodiments, the device 1 includes one or more of the weighted body feature, cooling feature, tactile feature and aromatherapy feature, and in a preferred exemplary embodiment includes all of these features.

In exemplary embodiments, the location of each feature may be selected so as to provide a even distribution of weight across the device 1, thereby allowing a user to feel comfort from the device 1 regardless of where the device 1 is in contact with the user.

In exemplary embodiments, one or more of the devices 1 may be made available in schools, private psychiatric offices, special needs facilities and elderly homes.

In exemplary embodiments, the device 1 may be made using patterning and sewing techniques, as is known in the art.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

1. A therapeutic device that combines a plurality of sensory features designed to reduce anxiety in children, adolescents and adults with and without special needs, comprising:
a main body configured to provide weighted pressure to a user of the device;
one or more components extending from the main body;
one or more sub-components extending from the one or more components, at least one of the main body, the one or more components and the one or more sub-components being stuffed with a filling;
a tactile feature disposed at or within the one or more components or the one or more sub-components; and
an aromatherapy feature disposed at or within one or more of the components or the one or more sub-components.

2. The therapeutic device of claim 1, wherein the cooling feature comprises a cool pack or an ice pack.

3. The therapeutic device of claim 1, wherein the tactile feature comprises a patch of rough material.

4. The therapeutic device of claim 1, wherein the tactile feature comprises beads.

5. The therapeutic device of claim 1, wherein the aromatherapy feature comprises one or more essential oils.

6. The therapeutic device of claim 1, wherein the therapeutic device is a stuffed toy.

7. The therapeutic device of claim 6, wherein the stuffed toy resembles an animal comprising at least one of a body, head, ears, legs, paws and tail.

8. The therapeutic device of claim 7, wherein the cooling feature is disposed within one of the ears.

9. The therapeutic device of claim 7, wherein the tactile feature is disposed within the tail.

10. The therapeutic device of claim 7, wherein the tactile feature is disposed at one of the ears.

11. The therapeutic device of claim 1, further comprising a cooling feature disposed at or within the one or more components or the one or more sub-components.
